(19) United States
(12) Patent Application Publication
(10) Pub. No.: US 2009/0280088 A1
(43) Pub. Date: Nov. 12, 2009

(54) Method of controlling hair shape

(75) Inventors: Akira Hachiya, Cincinnati, OH (US); Penkanok Sriwiriyanont, Cincinnati, OH (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Kao Corporation

(21) Appl. No.: 12/149,912

(22) Filed: May 9, 2008

(51) Int. Cl. C12Q 1/68; A61K 48/00; G01N 33/53; A61K 31/7088; A61P 17/00

(52) U.S. Cl. 424/93.2; 435/6; 436/501; 435/7.1; 514/44 R

(57) ABSTRACT

The present invention provides a method for determining or modulating hair morphology and properties of natural wavy and frizzy hair and a method for evaluating or selecting a hair morphology modulator. The present invention provides a method for modulating hair morphology, including controlling an expression of IGFBP-5 gene or IGFBP-5 protein in a human hair follicle.

### TECHNICAL FIELD

The present invention relates to a method for modulating hair morphology; for example, natural wavy or frizzy hair.

### BACKGROUND ART

Some people are troubled with their natural wavy and frizzy hair, and others want to wave straight hair. Hitherto, for straightening such natural wavy and frizzy hair or for waving straight hair, a hair-straightening agent or a permanent waving agent has been used to cleave a cystine bond in hair keratin, whereby hair is straightened or curled. However, use of such chemical agents damages hair to not a small extent.

Therefore, demand has arisen for development of a reliable method for straightening natural wavy or frizzy hair or for waving straight hair, without damaging hair.

Generally speaking, natural wavy hair is thought to be formed by factors including distribution of ortho- and para-cortex cells, quality and quantity of keratin-related protein, amount of disulfide bond of protein, transglutaminase and other differentiation-related enzymes present in a hair root, and shape of a hair bulb itself. A recent study has reported that insulin-like growth factor binding protein-5 (IGFBP-5), which is expressed in the medulla of a mouse, plays an important role in determining hair morphology (Schlake T. Development, 132: 2981, 2005, Schlake T. Mech. Dev. 122: 988, 2005).

In a human hair follicle, IGFBP-5 is expressed in the hair papilla and root sheath, and is a molecule playing an important role in reconstruction of an extracellular matrix (Firth S. M., Baxter R. C., Endocr. Rev. Dec. 23: 824, 2002; Batch J. A. et al., J. Invest. Dermatol. 106: 471, 1996). However, the relationship between IGFBP-5 and human hair morphology has not been reported. In general, hair structure and hair cycle considerably differ between mouse hair and human hair. The IGFBP-5 expression site of mice is the medulla, but in human the expression site is the hair papilla or outer root sheath. Therefore, the study of IGFBP-5 in mice cannot be extended to human cases.

### SUMMARY OF THE INVENTION

Accordingly, the present invention provides the following 1) to 4).

1) A method for modulating hair morphology, including controlling an expression of IGFBP-5 gene or IGFBP-5 protein in a human hair follicle.

2) A method for determining hair properties, including measuring and evaluating an expression level of IGFBP-5 gene or IGFBP-5 protein in a human hair follicle.

3) A method for evaluating or selecting a hair morphology modulator, wherein an expression level of IGFBP-5 gene or IGFBP-5 protein in a human hair follicle is employed as an index.

4) A method for evaluating or selecting a hair morphology modulator, including the following steps (A) to (D):

(A) a step of exposing a human hair follicle to a test substance;

(B) a step of measuring an expression level of IGFBP-5 gene or IGFBP-5 protein in the human hair follicle;

(C) a step of comparing the expression level of IGFBP-5 gene or IGFBP-5 protein as measured in step (B) with that of a control hair follicle which has not been exposed to the test substance; and

(D) a step of selecting, on the basis of the results obtained in step (C), the test substance that decreases or increases the expression level of IGFBP-5 gene or IGFBP-5 protein, as a hair morphology modulator.

### BRIEF DESCRIPTION OF THE DRAWINGS

FIG. 1: A graph showing IGFBP-5 gene expression in hair follicles of straight hairs and kinky hairs.

FIG. 2: Photographs showing immunohistological analysis of IGFBP-5 protein in hair follicles of straight hairs and kinky hairs.

FIG. 3: Photographs showing hair follicles isolated from kinky hairs and cultured in the absence of recombinant human IGFBP-5. In each photograph, hair follicles at the start of culture, and on day 3, day 5 and day 7 are given from left to right.

FIG. 4: Photographs showing hair follicles isolated from kinky hairs and cultured in the presence of recombinant human IGFBP-5. In each photograph, hair follicles at the start of culture, and on day 3, day 5 and day 7 are given from left to right.

FIG. 5: A graph showing variance of tangential angles of hair follicles from kinky hairs to which IGFBP-5 has been added (A), and a graph showing curvature of the same (B).

FIG. 6: Photographs showing the effect of IGFBP-5 on the extracellular matrix (ECM) of hair follicles from kinky hairs.

FIG. 7: Photographs showing significant increase in curvature of hair observed after transplantation of hair follicles derived from straight hairs (straight) or curly hairs (curly) in which IGFBP-5 has been over-expressed.

FIG. 8: Photographs showing hair regenerated with severe kinkiness caused by over-expression of IGFBP-5.

FIG. 9: SEM images of hair regenerated from hair follicles of straight hairs in which IGFBP-5 has been over-expressed.

### DETAILED DESCRIPTION OF THE INVENTION

The present invention is directed to a method for determining or modulating hair morphology and properties such as natural wavy and frizzy hair and to a method for evaluating or selecting a hair morphology modulator.

The present inventors have investigated factors relating to human hair morphology using molecular biological techniques, and have found that IGFBP-5 gene and IGFBP-5 protein are expressed at high levels in hair follicles of subjects with frizzy hair.

According to the present invention, hair morphology can be modulated without damaging hair. That is, for example, natural wavy or frizzy hair can be straightened, straight hair can be waved, or waving can be promoted. Also, hair morphology or properties such as natural wavy and frizzy hair can be determined. In addition, a material such as a drug or a cosmetic composition for modulating hair morphology can be evaluated or selected.

In the present invention, the term “IGFBP-5” refers to insulin-like growth factor binding protein-5, and the term “IGFBP-5 gene” refers to a gene encoding IGFBP-5. IGFBP-5 was first cloned in 1994, and the complete nucleotide sequence has been reported (Allander S. V., Larsson C., Ehrenborg E., Suwanichkul A., Weber G., Morris S. L., Bajalica S., Kiefer M. C., Luthman H., Powell D. R., Characterization of the chromosomal gene and promoter for human insulin-like growth factor binding protein-5. J. Biol. Chem. Apr. 8, 1994; 269(14): 10891-8).

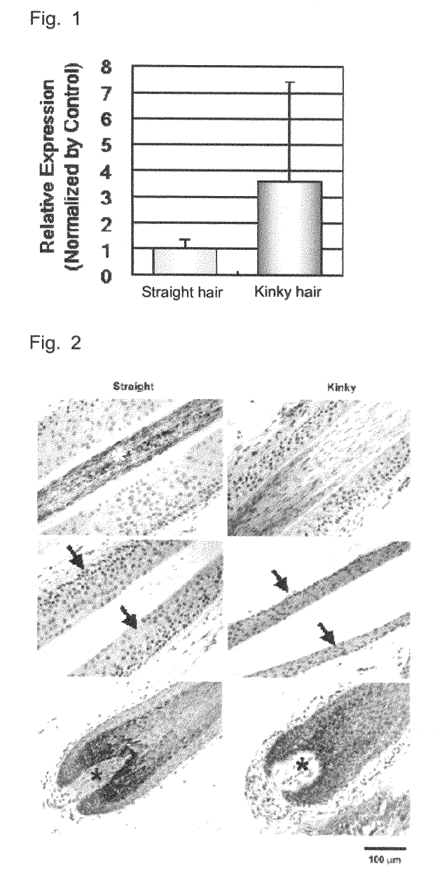

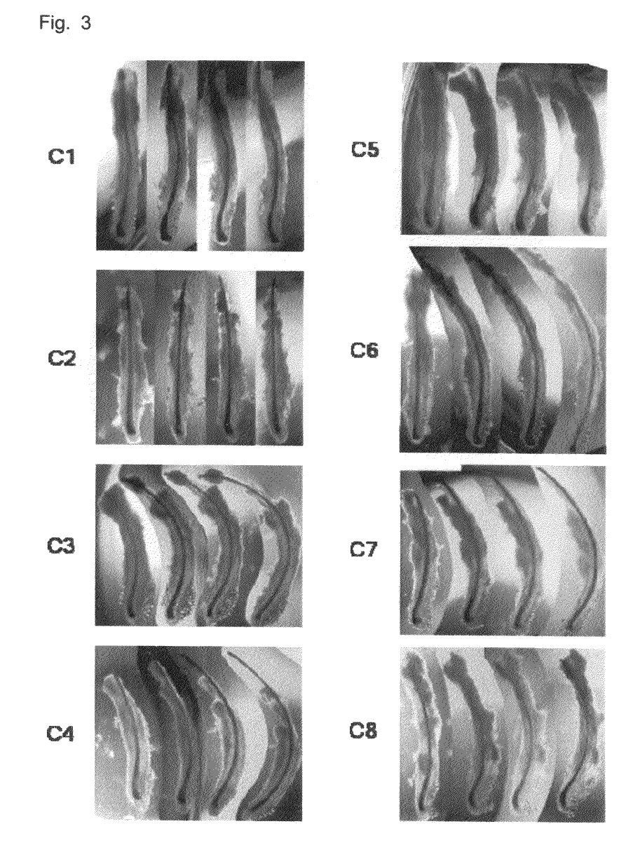

As described in the Example hereinbelow, hair follicles are removed from a Caucasian subject with straight hair and a subject of African descent with kinky hair, and the feature of IGFBP-5 gene expression was compared. As a result, the expression level of IGFBP-5 gene in hair follicles of kinky hairs was found to be about 3.5 times higher compared to that in hair follicles of straight hairs (FIG. 1). In an immunohistological analysis, positive areas attributed to IGFBP-5 were observed in the outer root sheath (ORS). In the hair follicle of kinky hair, IGFBP-5 was expressed asymmetrically (arrows in FIG. 2).

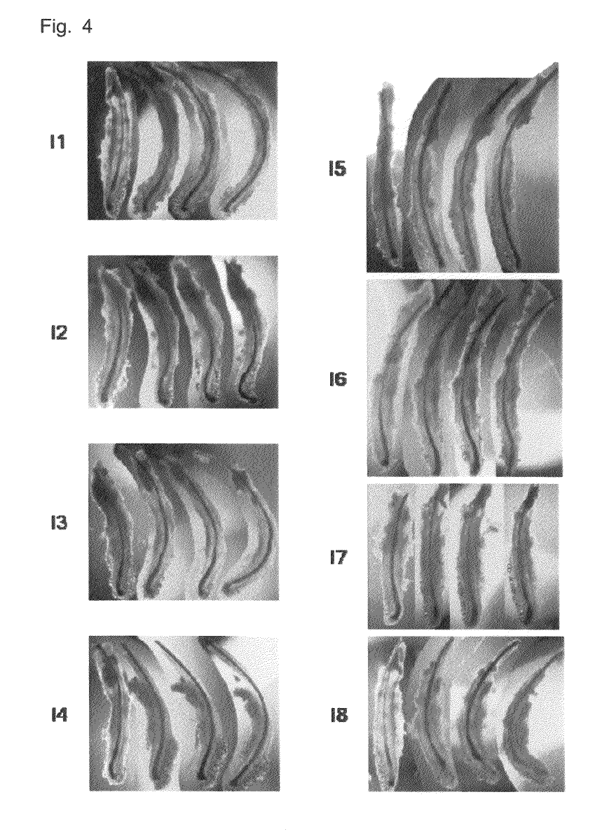

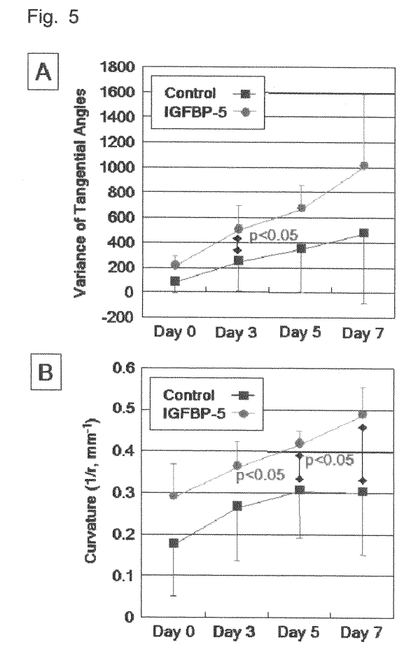

Hair follicles of kinky hairs were removed from African descents and cultured in the presence or absence of recombinant human IGFBP-5, and the change in curving degree was investigated. As a result, the hair follicles grew with kinkiness even in the absence of IGFBP-5, but the curving was more promoted in the presence of IGFBP-5 (FIGS. 3 and 4). In addition, waving degree (frequency and degree of curving) and general curving degree (curving strength) were significantly increased in the presence of IGFBP-5 (FIGS. 5A and 5B).

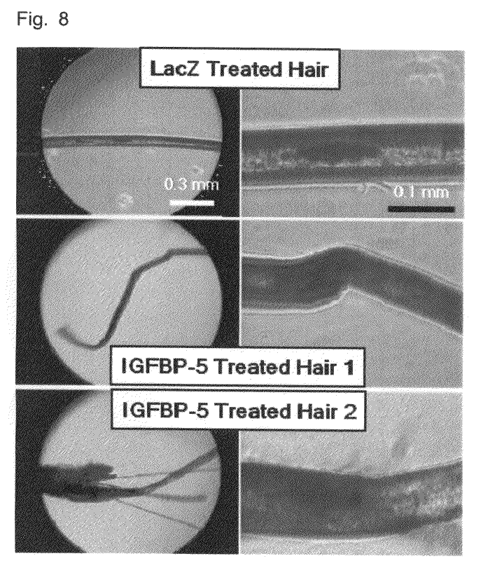

In another experiment, a hair follicle of straight hair in which IGFBP-5 gene had been over-expressed was transplanted into an immunodeficient mouse. In this case, the regenerated hair was kinking as if natural wavy hair had been transplanted. In some cases, kinky hairs were observed (FIGS. 8B, 8C, and 9). When a hair follicle of natural wavy hair in which IGFBP-5 gene had been over-expressed was transplanted, curving degree was further increased (FIGS. 8E and 8F).

As described above, an expression product of IGFBP-5 (i.e., IGFBP-5 protein) can be considered a factor relating to human hair morphology. Therefore, through controlling expression of IGFBP-5 and IGFBP-5 gene in a human hair follicle, hair morphology can be modulated. Furthermore, through measuring and evaluating the expression levels, human hair morphology and properties can be determined, and a hair morphology modulator can be screened.

In the present invention, no particular limitation is imposed on the human race whose hair morphology is modulated, determined, or evaluated. However, people of African descent are preferred, since high expression levels of IGFBP-5 gene and IGFBP-5 protein can be obtained in hair follicles; the morphology of natural wavy hair is thought to greatly depend on the expression; and many of them care about kinky hair.

### 1) Method of Modulating Hair Morphology

The method for modulating hair morphology according to the present invention is carried out by controlling an expression of IGFBP-5 gene or IGFBP-5 protein in a human hair follicle.

For example, for those who want to straighten their natural wavy or frizzy hair, such hair can be suppressed by inhibiting an expression of IGFBP-5 gene or IGFBP-5 protein, whereas for those who want to wave hair, waving can be realized or promoted by inducing or promoting an expression of IGFBP-5 gene.

For example, for inhibiting the expression of IGFBP-5 gene, the expression level of IGFBP-5 gene in a human hair follicle can be suppressed to an mRNA expression level in a human with straight hair or lower. In order to promote waving, the IGFBP-5 gene expression level can be adjusted to a level higher than an mRNA expression level in a human with straight hair, for example, to about 3 to 10 or more times the mRNA expression level.

Suppression, induction, or promotion of expression of IGFBP-5 gene or IGFBP-5 protein in a human hair follicle may be performed through a generally employed technique. For example, suppression of the gene expression can be accomplished by methods using antisense nucleotides; e.g., by inhibiting translation of mRNA. Induction or promotion of the gene expression can be accomplished by expressing the IGFBP-5 gene using gene transfer technique such as a virus vector. Basically, the expression of IGFBP-5 protein can be suppressed by suppressing the expression of IGFBP-5 gene, and can be induced or promoted by intracutaneously injecting a recombinant human IGFBP-5 to elevate the amount of IGFBP-5 protein.

### 2) Method of Determining Hair Properties

The method for determining hair properties according to the present invention is carried out through measuring and evaluating an expression level of IGFBP-5 gene or IGFBP-5 protein in a human hair follicle.

In one possible method, hair follicles are removed from the scalp; the presence of the hair bulb above the hair papilla in a hair follicle of interest is checked; and the expression level of IGFBP-5 gene or IGFBP-5 protein in the hair follicle is determined through the RT-PCR method, an immunohistological method employing an anti-IGFBP-5 antibody or the like, or western blotting. Alternatively, instead of removed hair follicles, scalp pieces obtained through punch biopsy may be evaluated through an immunohistological technique. Also, hair follicles may be isolated from the scalp pieces, and the isolated hair follicles are evaluated through the above technique.

As used herein, the term “hair properties” refers to properties such as easiness of hair bundling, elasticity, and easiness of cutting. According to the present invention, the hair properties are determined through measuring and evaluating an expression level of IGFBP-5 gene or IGFBP-5 protein in a human hair follicle.

### 3) Method of Evaluating or Selecting a Hair Morphology Modulator

The method of the present invention for evaluating or selecting a hair morphology modulator is performed through employing an expression level of IGFBP-5 gene or IGFBP-5 protein in a human hair follicle as an index; i.e., measuring the expression level of IGFBP-5 gene or IGFBP-5 protein in the human hair follicle and comparing the obtained data.

Specifically, the method for evaluating or selecting a hair morphology modulator is carried out through the following steps (A) to (D):

(A) a step of exposing a human hair follicle to a test substance;

(B) a step of measuring an expression level of IGFBP-5 gene or IGFBP-5 protein in the human hair follicle;

(C) a step of comparing the expression level of IGFBP-5 gene or IGFBP-5 protein as measured in step (B) with that of a control hair follicle which has not been exposed to the test substance; and

(D) a step of selecting, on the basis of the results obtained in step (C), the test substance that decreases or increases the expression level of IGFBP-5 gene or IGFBP-5 protein, as a hair morphology modulator.

The hair follicles employed in the invention refer to a tissue surrounding the entirety of the hair root collected from the skin of human; specifically, the entirety of the tissue including the hair shaft, hair papilla, hair matrix cells, and hair root.

Exposure of a hair follicle to a test substance may be performed through the following procedures. Specifically, the test substance is added in advance to a culture solution at a predetermined concentration and, subsequently, the hair follicle is placed in the culture solution. Alternatively, the hair follicle is placed in a culture solution, and the test substance is added to the culture solution at a predetermined concentration.

Organ culturing of human hair follicles may be performed through a known hair follicle organ culturing method (Philpott M. P., Sanders D., Westgate G. E., Kealey T., Human hair growth in vitro: a model for the study of hair follicle biology, J. Dermatol. Sci. July 1994; 7 Suppl.: S55-72). For example, collected hair follicles are placed in a 24-well plate to which a culture solution has been added, followed by culturing at 37° C. in air containing CO₂ generally for 2 to 56 days, preferably 4 to 21 days.

Examples of the culture solution include an RPMI 1640 medium, a William's E medium, and a DMEM/HamF12 (1:1) medium. Agar or gelatin may be appropriately added to the culture solution. If required, an antibiotic substance, amino acid, blood serum, a growth factor, a biological extract, or the like may be added thereto.

The substance that decreases the expression level of IGFBP-5 gene refers to a substance which suppresses expression or promotes decomposition of mRNA complementary to the polynucleotide of the gene, and the substance that decreases the expression level of IGFBP-5 protein refers to a substance which suppresses expression or promotes decomposition of IGFBP-5 gene or IGFBP-5 protein, whereby the expression level of IGFBP-5 protein is decreased.

The substance that decreases the expression level of IGFBP-5 gene or IGFBP-5 protein serves as an agent for straightening natural wavy or frizzy hair, whereas the substance that induces or promotes expression of IGFBP-5 gene or IGFBP-5 protein serves as a waving promoter. Such a hair morphology modulator can provide a pharmaceutical product, cosmetic composition, etc. for straightening natural wavy or frizzy hair or promoting waving through administration to human.

Detection of the gene expression and determination of the expression level may be performed through a known method such as RT-PCR employing RNA prepared from the aforementioned tissue and a complementary polynucleotide transcribed from the RNA.

The expression level of IGFBP-5 protein may be determined through, for example, a known method such as western blotting employing, for example, an antibody that recognizes the IGFBP-5 protein (e.g., anti-IGFBP-5 antibody).

Western blotting can be carried out by using a primary antibody which recognizes the expression product, and a secondary antibody which binds to the primary antibody and which is labeled with a radioisotope (e.g., ¹²⁵I), a fluorescent substance, or an enzyme (e.g., horseradish peroxidase (HRP)); and by measuring a signal from such a labeling substance by means of, for example, a radiation meter or a fluorescence detector.

The antibody may be a monoclonal antibody or polyclonal antibody prepared by using the expression product as an immunogen. The antibody may be a commercial antibody which is guaranteed to recognize the expression product, or may be prepared through immunization of an animal (e.g., rabbit or mouse).

### EXAMPLES

### (1) Preparation of Human Hair Follicles

According to a protocol approved by the Institutional Review Board of Cincinnati Children's Hospital Medical Center, two scalp tissue pieces were obtained from each of a Caucasian subject with straight hair, an African descent subject with kinky hair, a Hispanic subject with curly hair, with informed consent, through punch biopsy (diameter of punch: 6 mm). The tissue pieces were transported while they were immersed in DMEM containing an antibacterial agent at 4° C. Within two hours of the collection of the tissue pieces, adipose tissue was removed from each tissue piece to a maximum degree under a dissecting microscope, to thereby isolate hair follicles, which were subsequently employed in the experiments hereinbelow.

### (2) Analysis of Expression of IGFBP-5 in Straight Hair and Kinky Hair

Hair follicles were removed from a Caucasian subject with straight hair and from an African descent subject with kinky hair. In order to avoid decomposition of RNA, the follicles were immersed in an RNAlater (product of Qiagen, Valencia, Calif.) immediately after collection, and total RNA was collected by means of an RNeasy micro kit (product of Qiagen). cDNA was synthesized through a routine method employing oligo dT and Moloney murine leukemia virus reverse transcriptase. For investigating the difference in IGFBP-5 expression level between straight hair and kinky hair, a probe specific to the gene and TaqMan Gene Expression Assays (Assay ID: Hs00181213_ml) were purchased from Applied Biosystems (Foster City, Calif.). Real-time quantitative RT-PCR was performed through employment of ABI PRISM 7300 sequence detection system (product of Applied Biosystems). Expression of glyceraldehyde-3-phosphate dehydrogenase (GAPDH Assay ID: human GAPDH VIC-MGB No. 4326317E) was employed as the internal standard. Separately, for investigating the expression site of IGFBP-5 protein in a hair follicle, scalp pieces of a Caucasian subject and an African descent subject were immunohistologically analyzed. Specifically, each scalp piece was fixed with formalin and embedded in paraffin. Six-micrometer-thick sections were prepared through cutting the paraffin block, then deparaffined, hydrated, subjected to removal of endogenous peroxidase activity, and subjected to blocking reaction. The thus-treated sections were incubated with a 5-μg/mL rabbit polyclonal IGFBP-5 (product of Acris Antibodies GmbH, Herford, Germany), and subsequently reacted with a biotinylated secondary antibody. Finally, a biotinylated secondary antibody was recognized by use of streptavidin-bound HRP based on the specific binding between streptavidin and biotin. Positive areas in which rabbit polyclonal IGFBP-5 recognizes were visualized by use of 3-amino-9-ethylcarbazole (product of OmniTaqs Plus Universal-AEC, Thermo Shandon, Pa.) as an HRP substrate. Instead of rabbit polyclonal IGFBP-5, normal rabbit IgG (product of Vector Laboratories, Burlingame, Calif.) was employed as a negative control.

The gene expression level was relatively evaluated to the gene expression level of IGFBP-5 in a hair follicle of straight hair as a value of 1. The gene expression level of IGFBP-5 in a hair follicle of kinky hair was about 3.5, which is explicitly higher than the expression level in the follicle of straight hair (FIG. 1). In the immunohistological analysis, positive areas attributed to IGFBP-5 were observed in the outer root sheath (ORS), and IGFBP-5 was expressed asymmetrically in the hair follicle of kinky hair (arrows in FIG. 2). Although a previous report discloses that IGFBP-5 gene is expressed in the hair papilla (Batch J. A. et al., J. Invest. Dermatol. 106: 471, 1996), in the present analysis, no positive area was observed in the hair papilla (as indicated by asterisks in FIG. 2).

(3) Organ Culture of Hair Follicles Isolated from Kinky Hair in the Presence of Recombinant Human IGFBP-5

Recombinant human IGFBP-5 (product of Novozymes GroPep, Thebarton, SA, Australia) (250 ng/mL) was added to a culture medium for organ culture of hair follicles (phenol red free Williams E medium (product of Sigma, St. Louis, Mo.) supplemented with 2 mM L-glutamine (product of Invitrogen, Calsbard, Calif.), 10-μg/mL transferrin (product of Invitrogen), 10-ng/mL sodium selenate (product of Sigma), 10-ng/mL hydrocortisone (product of Invitrogen), and antibiotics antimycotics (product of Invitrogen)). The mixture was cultured at 37° C. under an atmosphere containing 5% CO₂. On day 3 and day 5 from the start of culturing, the medium was replaced, and hair follicles were collected on day 7. Photoimages were taken at the start of culture, and on day 3, day 5 and day 7.

The curving degree of the samples was analyzed with reference to the photographs taken. Specifically, the middle line of each hair follicle observed in each photograph was traced to form a line, and variance of tangential angle and curvature (1/r) were calculated by analysis software.

FIG. 3 shows changes over time of eight hair follicles from kinky hairs (C1 to C8) obtained from an African descent subject and cultured in the absence of IGFBP-5, and FIG. 4 shows changes over time of eight hair follicles from kinky hairs (I1 to I8) obtained from the same subject and cultured in the presence of IGFBP-5. In each photograph, hair follicles at the start of culture, and on day 3, day 5 and day 7 are given from left to right. Even in the absence of IGFBP-5, the hair follicles grew with kinking, but the curving was more promoted in the presence of IGFBP-5. The curving degree was quantitatively evaluated. The variance of tangential angle, which reflects waving degree (frequency and degree of curving), increased as time elapsed even in the absence of IGFBP-5. In the presence of IGFBP-5, the increase was found to be significantly (p<0.05) promoted particularly after culture day 3 (FIG. 5A). Similarly, the curvature, which reflects general curving degree (curving strength), increased as time elapsed even in the absence of IGFBP-5. However, through addition of IGFBP-5, the increase was found to be significantly (p<0.05) promoted particularly after culture day 5 and day 7 (FIG. 5B).

### (4) Effect of IGFBP-5 on Extracellular Matrix (ECM)

One of the functions of IGFBP-5 which are independent from those of IGF-I has been reported to be strong interaction with an extracellular matrix (ECM) (Firth S. M., Baxter R. C., Endocr. Rev. 6: 824-854, 2002). Thus, the mechanism of promoting kinky hair by IGFBP-5 was investigated. Specifically, after 7-day organ culturing, frozen sections were prepared. Expression of collagen IV, which is a type of ECM, was investigated by use of an antibody specific for the protein. More specifically, isolated hair follicles which had been organ-cultured for seven days were immersed in 1% buffered paraformaldehyde overnight at 4° C. Subsequently, free aldehyde groups were blocked with 10 mM ethanolamine, and frozen in a Tissue-Tek OCT compound (product of Sakura Finetek, Torrance, Calif.), to thereby prepare the frozen sections. A mouse monoclonal antibody (product of EMD Biosciences, Inc. San Diego, Calif.) to collagen IV and a rabbit polyclonal antibody (product of Lab Vision Corporation) to loricrin were employed as primary antibodies. The primary antibodies were labeled with Alexa Flour 546 anti-rabbit (product of Invitrogen) and Alexa Flour 488 anti-mouse (product of Invitrogen), and the sections were analyzed under a Zeiss LSM510 confocal microscope (product of Zeiss, Thornwood, N.Y.). Counter staining was performed by use of Draq-5 (product of Alexis Corporation, Lausen, Switzerland).

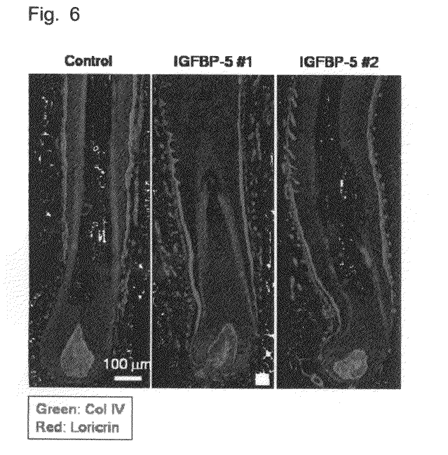

As shown in FIG. 6, as compared with the control to which IGFBP-5 had not been added, collagen IV in the IGFBP-5-treated hair follicle was found to greatly intrude toward the ORS basement membrane, particularly in a curved portion of the hair follicle.

### (5) Transferring IGFBP-5 Gene to Hair Follicles

Transfer of IGFBP-5 gene into hair follicles was performed by use of a VSV-G pseudo-type lentivirus vector. CMV was employed as a promoter for expressing IGFBP-5. The IGFBP-5 gene was cloned through amplification through PCR from human skin cDNAs (product of Invitrogen) by use of the following primers:

5′: ATATATCTAGAGCCACCATGGTGTTGGTCACCGC
3′: ATATAGGATCCCTCAACGTTGCTGTC.

After confirmation of the sequence of the cloned gene, the gene was transferred into a transfer vector for lentivirus. Subsequently, the corresponding virus vector was produced through a method disclosed in U.S. patent Ser. No. 12/010426. In order to over-express IGFBP-5, isolated hair follicles were immersed in a lentivirus titer liquid (1.6×10⁹ TU/mL) for four hours and then, cultured in a hair follicle organ culturing medium (phenol red free Williams E medium (product of Sigma, St. Louis, Mo.) supplemented with 2 mM L-glutamine (product of Invitrogen, Calsbard, Calif.), 10-μg/mL insulin (product of Invitrogen), 10-μg/mL transferrin (product of Invitrogen), 10-ng/mL sodium selenate (product of Sigma), 10-ng/mL hydrocortisone (product of Invitrogen), and antibiotics antimycotics (product of Invitrogen)) at 37° C. under an atmosphere containing 5% CO₂. Nineteen hours after the start of culturing, the hair follicles were immersed in the same titer solution for second transfection. As a control with respect to the IGFBP-5 gene, a β-galactosidase (LacZ) gene was employed. A lentivirus titer solution for expressing with a CMV promoter was produced, and the LacZ gene was treated in the same manner.

After transfection with the lentivirus, the hair follicles were transplanted to immunodeficient (severe combined immunodeficient; SCID) mice, and the hair morphology of the regenerated hair was evaluated. Specifically, a hole (diameter: about 1 mm, depth: about 5 to 20 mm) was made by means of a 18G syringe (diameter: 1 mm) through the back skin to the fascia of each SCID mouse such that the hole axis is parallel to the mousers hair shaft. A medium for hair follicle organ culture (about 25 μL) was injected in the hole, and a hair follicle was inserted into the hole. The inserted hair follicle was immobilized to the skin by use of an instantaneous adhesive (superglue) for biological use. To the insertion site, 2.5% lidocaine was added dropwise for local anesthesia, and the entire back of the mouse including the transplant site was covered with adhesive film (product of Tegaderm, 3M, Ontario, Canada). The film was confirmed to be adhered to the back of the mouse for at least 48 hours.

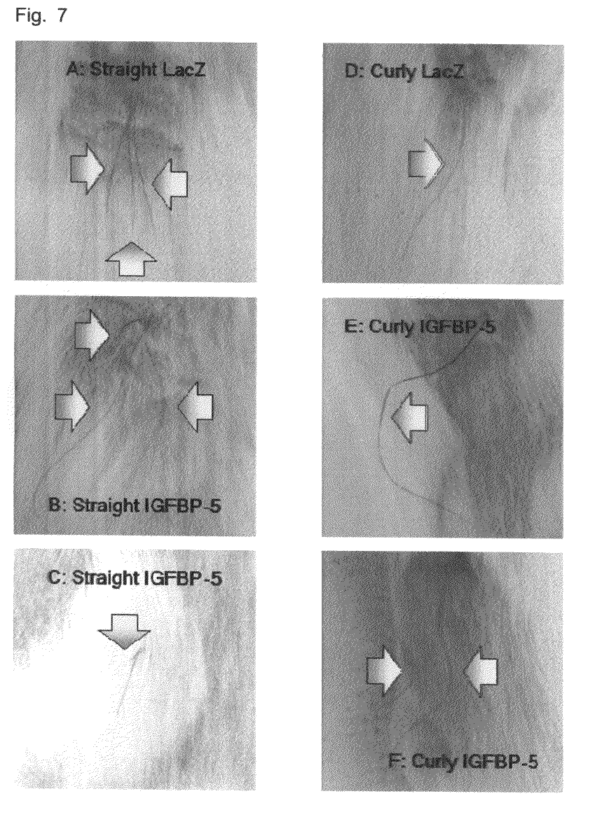

When a hair follicle in which β-galactosidase had been over-expressed was transplanted, hair grown several months after transplantation maintained the hair morphology of the hair follicle donor, regardless of the type of hair (straight or curly) (FIGS. 7A and 7D). In contrast, when a hair follicle from straight hair in which IGFBP-5 had been over-expressed was transplanted, regenerated hair had such morphology that a curly hair had been transplanted, and some hair filaments were found to be remarkably curled (FIGS. 7B, 7C, and 8). When a hair follicle from curly hair in which IGFBP-5 had been over-expressed was transplanted, the curving degree was considerably increased (FIGS. 7E and 7F).

Regenerated hair was analyzed though SEM. Specifically, a regenerated hair filament was cut to pieces of 2 to 3 mm, and each piece was fixed on an aluminum specimen mount (product of Electron Microscopy Sciences, Hatfield, Pa.). The shape of a hair shaft and the cross-section of the hair filament were visualized through two minutes-coating with gold-palladium under evaporation by means of a Hummer Sputter Coater (product Anatech, Hayward, Calif.). The images were obtained by means of an FEI Quanta 200 SEM (product of FEI, Hillsboro, Oreg.) at an operating voltage of 20 kV.

As shown in FIG. 9, the hair regenerated from a straight hair from a Caucasian subject in which follicle β-galactosidase had been over-expressed was found to have a structure similar to that of the hair of the hair follicle donor. Specifically, thin flakey cuticles were overlapped each other about 4/5 portions thereof, which form an irregular but dense, and transversely undulating pattern. The hair regenerated from a hair follicle of the same subject in which follicle IGFBP-5 had been over-expressed was also observed under an SEM. In this case, cuticles were provided at low density and did not regularly undulate. The morphology was very similar to that of kinky hair of an African descent subject (Guohua Wei, Bharat Bhushan, Peter M. Torgerson, Ultramicroscopy 105: 248-266, 2005).

1. A method for modulating hair morphology, comprising controlling an expression of IGFBP-5 gene or IGFBP-5 protein in human hair follicle.

2. A method for determining hair properties, comprising measuring and evaluating an expression level of IGFBP-5 gene or IGFBP-5 protein in human hair follicle.

3. The method for modulating hair morphology as described in claim 1 or the method for determining hair properties as described in claim 2, wherein the human is a subject of African descent.

4. A method for evaluating or selecting a hair morphology modulator, wherein an expression level of IGFBP-5 gene or IGFBP-5 protein in a human hair follicle is employed as an index.

5. A method for evaluating or selecting a hair morphology modulator, comprising the following steps (A) to (D):
(A) a step of exposing a human hair follicle to a test substance, wherein IGFBP-5 gene or IGFBP-5 protein has been expressed in the human hair follicle;
(B) a step of measuring an expression level of IGFBP-5 gene or IGFBP-5 protein in the human hair follicle;
(C) a step of comparing the expression level of IGFBP-5 gene or IGFBP-5 protein as measured in step (B) with that of a control hair follicle which has not been exposed to the test substance; and
(D) a step of selecting, on the basis of the results obtained in step (C), the test substance that decreases or increases the expression level of IGFBP-5 gene or IGFBP-5 protein, as a hair morphology modulator.

6. The method as described in claim 4 or 5, wherein the human is a subject of African descent.


SEQUENCE LISTING (2 sequences)

SEQ ID NO: 1 - DNA, 34 bp, Artificial Sequence
  Synthetic Oligonucleotide as PCR primer designed from nucleotide sequence of IGFBP-5 gene
atatatctagagccaccatggtgttggtcaccgc

SEQ ID NO: 2 - DNA, 26 bp, Artificial Sequence
  Synthetic Oligonucleotide as PCR primer designed from nucleotide sequence of IGFBP-5 gene
atataggatccctcaacgttgctgtc